(19) United States
(12) Patent Application Publication
(10) Pub. No.: US 2024/0099598 A1
(43) Pub. Date: Mar. 28, 2024

(54) WEARABLE ELECTRONIC DEVICE, DETERMINATION STANDARD SETTING METHOD AND STORAGE MEDIUM

(71) Applicant: CASIO COMPUTER CO., LTD., Tokyo (JP)

(72) Inventors: Tomoo KASUO, Tokyo (JP); Takeshi MIYAKE, Tokyo (JP); Keiichi NOMURA, Uenohara-shi (JP)

(73) Assignee: CASIO COMPUTER CO., LTD., Tokyo (JP)

(21) Appl. No.: 18/370,317

(22) Filed: Sep. 19, 2023

(30) Foreign Application Priority Data
Sep. 26, 2022 (JP) 2022-152140

(51) Int. Cl. A61B 5/024; A61B 5/00

(52) U.S. Cl. CPC A61B 5/02416; A61B 5/02438; A61B 5/74

(57) ABSTRACT

A wearable electronic device includes, a light emitter that emits light with a certain wavelength toward a body while the device is worn on the body; a light receiver that measures an incident light amount of the light with the certain wavelength; a posture measurer that measures a posture of a part of the body where the device is worn; and one or more processors. The processor is configured to, determine a posture of the part of the body where the device is worn based on a measured result measured by the posture measurer, and set a first standard value for determining whether the device is worn on the body or not based on the incident light amount in a case that the part of the body where the device is worn is in a standard posture.

### BACKGROUND OF THE INVENTION

### Field of the Invention

The present disclosure relates to a wearable electronic device, a determination standard setting method, and storage medium storing a program.

### Description of the Related Art

There are electronic devices used by being worn on a body such as an arm. If the electronic device is used to measure vital information of the body on which the device is worn, there is no meaning in an operation of measuring when a user is not wearing the device. Therefore, in such electronic devices, the device may stop measuring or may discard the result of measurement after determining whether the device is being worn on the body.

JP 2018-050743A discloses an electronic device that is worn on the arm to measure a pulse. The light used to measure the pulse is used to determine whether the device is being worn.

### SUMMARY OF THE INVENTION

According to one aspect of the present disclosure, a wearable electronic device includes: a light emitter that emits light with a certain wavelength toward a body while the device is worn on the body; a light receiver that measures an incident light amount of the light with the certain wavelength; a posture measurer that measures a posture of a part of the body where the device is worn; and one or more processors. The processor is configured to, determine a posture of the part of the body where the device is worn based on a measured result measured by the posture measurer, and set a first standard value for determining whether the device is worn on the body or not based on the incident light amount in a case that the part of the body where the device is worn is in a standard posture.

Further features of the present invention will become apparent from the following description of exemplary embodiments (with reference to the attached drawings).

### BRIEF DESCRIPTION OF THE DRAWINGS

FIG. 1 is a block diagram showing a functional configuration of a smart watch.

FIG. 2 is a bottom view showing a rear side of the smart watch.

FIG. 3A is a diagram to describe an example of detecting incident light amount.

FIG. 3B is a diagram to describe an example of detecting incident light amount.

FIG. 4 is a flowchart showing a determination standard value setting control process.

FIG. 5 is a flowchart showing another example of the determination standard value setting control process.

FIG. 6 is a flowchart showing a determination standard value adjustment control process.

### DESCRIPTION OF THE EMBODIMENTS

Hereinafter, embodiments of the present disclosure are described with reference to the drawings.

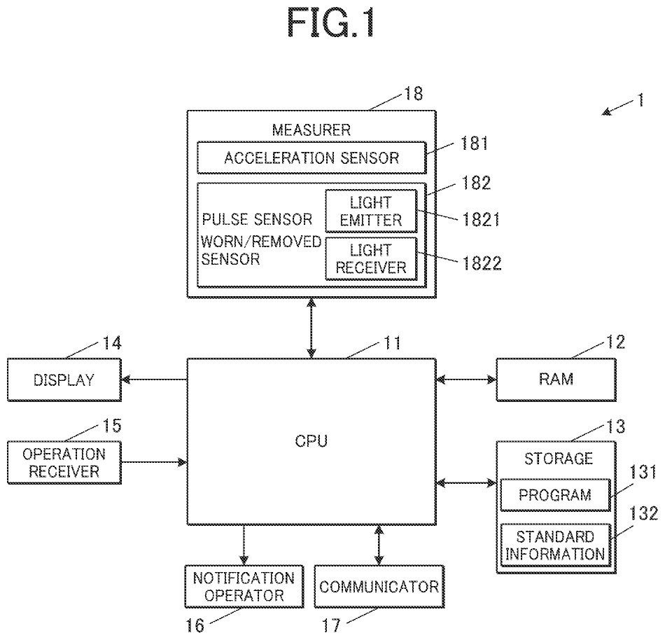

FIG. 1 is a block diagram showing a functional configuration of a smart watch 1, which is a wearable electronic device according to the present embodiment.

The smart watch 1 includes a CPU 11 (Central Processing Unit, controller), a RAM 12 (Random Access Memory), a storage 13, a display 14, an operation receiver 15, a notification operator 16, a communicator 17, and a measurer 18.

The CPU 11 is a processor that performs arithmetic processing and that centrally controls the operation of the smart watch 1. The CPU 11 may include a single processor, or may include a plurality of processors that perform arithmetic processing parallel or individually according to the intended use. The CPU 11 is not limited to a general purpose unit. The CPU 11 may be designed and manufactured specifically for a specific application.

The RAM 12 provides a working memory space for the CPU 11 and stores temporary data.

At least the CPU 11 and the RAM 12 are included in the computer of the smart watch 1 according to the present embodiment.

The storage 13 is a non-volatile memory and stores a program 131 and setting data. For example, the non-volatile memory is a flash memory, but is not limited to this. The setting data includes standard information 132 to determine whether or not the user is wearing the smart watch 1 on the arm (wrist) of the user as described later.

For example, the display 14 includes a digital display screen and is able to display various letters and figures based on control by the CPU 11. The digital display screen includes a liquid crystal display screen (LCD), for example.

The operation receiver 15 receives operation of input from outside and outputs to the CPU 11 an operation signal according to the received input operation. The input operation is received by an operation receiving member, for example, a push-button switch or a crown. Alternatively, the smart watch 1 may include a touch panel that is positioned overlapped on the digital display screen as the operation receiving member.

The notification operator 16 performs a notification operation to the user. The notification operation may be performed by generating a beep sound, a vibration, or the like, for example. Therefore, for example, the notification operator 16 may include a piezoelectric element overlapped on a diaphragm, a motor with a weight, or the like.

The communicator 17 controls transmission and reception of data to and from the external device. The communicator 17 is able to control transmission and reception of data according to communication standards (protocol) such as short range wireless communication (for example, Bluetooth (registered trademark)) or wireless LAN. The communicator 17 includes an antenna that transmits and receives radio waves with the communication frequency regarding the communication standards.

The measurer 18 measures a physical quantity and outputs the measured result to the CPU 11. The measurer 18 is configured to be capable of digitally converting the measured result, molding the data into an appropriate format, and outputting the data to the CPU 11.

The measurer 18 includes an acceleration sensor 181 (posture measurer) and pulse sensor 182. The pulse sensor 182 also functions as a worn/removed state detection sensor. The acceleration sensor 181 measures the acceleration in 3-axis directions such as 2-axis directions which are directions orthogonal to each other in a plane parallel to the display screen and 1-axis direction orthogonal to the plane. The acceleration to be measured includes gravitational acceleration. Therefore, by specifying the gravitational acceleration direction, a posture of the smart watch 1 can be estimated. In a state in which the user wears the smart watch 1 on the arm, the posture of the user's arm (part where device is worn) can be measured (estimated). The smart watch 1 can be worn on either the left or the right arm of the user. Therefore, the information showing which arm the device is worn on can be obtained as necessary according to operation of input on the operation receiver 15.

The pulse sensor 182 measures the pulse of the user wearing the smart watch 1 and outputs the measured result to the CPU 11. The pulse sensor 182 includes a light emitter 1821 and a light receiver 1822.

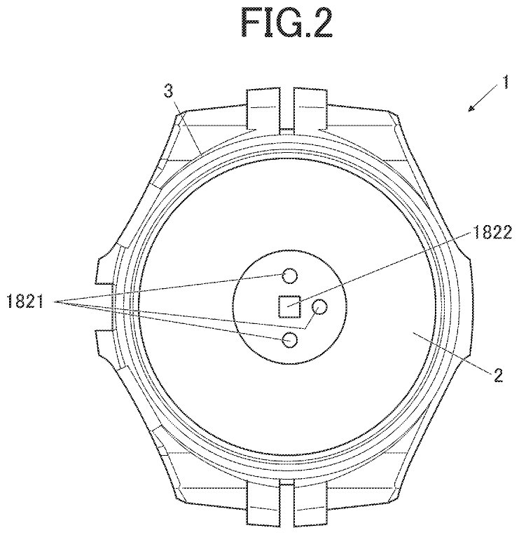

FIG. 2 is a bottom view showing a rear side of the smart watch 1.

One end of a tubular case 3 of the smart watch 1 is sealed with a back cover 2. The light emitter 1821 and the light receiver 1822 are positioned on the side where the back cover 2 of the smart watch 1 is (surface in contact with the body of the user). Therefore, the light emitter 1821 and the light receiver 1822 face the arm of the user when the smart watch 1 is attached to the user's arm. The light emitter 1821 generates light with a wavelength suitable for measuring the pulse, for example, green color light, and emits the light. With relation to the light emitter 1821, the light receiver 1822 is provided in a position where the green color light emitted from the light emitter 1821 and reflected inside the arm (in the body) enters, and measures an incident light amount of the green color light (light with a certain wavelength).

The pulse sensor 182 may be configured to be able to measure arterial blood oxygen saturation (SpO2) also. In this case, the pulse sensor 182 may be configured to be able to emit and detect a red color light in addition to the green color light.

On the other hand, the pulse sensor 182 also operates as the worn/removed state detection sensor. In a state in which the smart watch 1 is not worn on the arm, the emitted green color light is not reflected, and the incident light amount is greatly reduced. Therefore, an absolute value of the incident light amount can be used to determine whether the smart watch 1 is worn on the arm.

As the worn/removed state detection sensor, the light emitter 1821 may be configured to be able to emit infrared light (IR). If visible light is emitted when the smart watch 1 is not worn, this may expose the surroundings, and cause unintended influence such as being too bright. Therefore, infrared light may be used when it is determined whether the smart watch 1 is worn when the smart watch 1 is not worn.

Next, the operation of determining whether the smart watch 1 is worn is described.

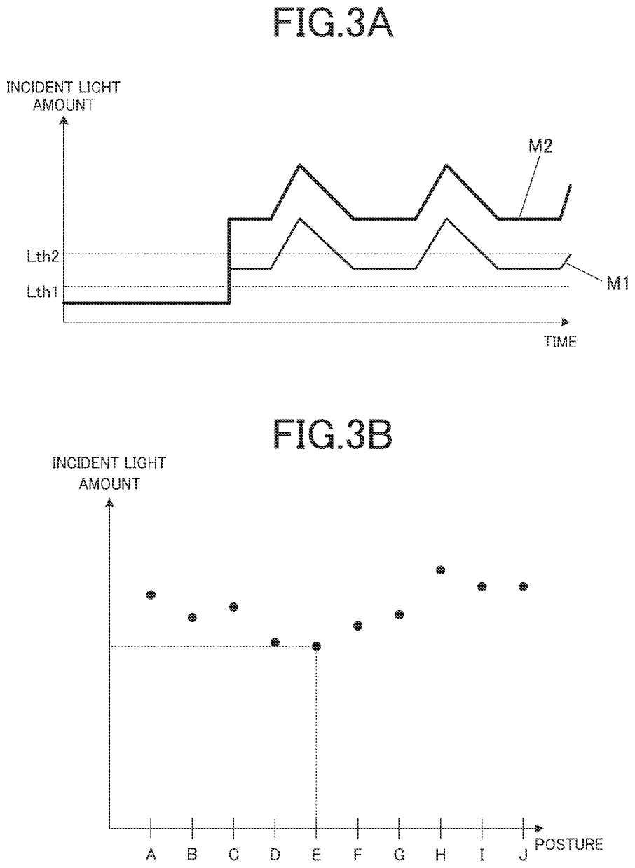

FIG. 3A and FIG. 3B are diagrams describing an example of detecting the incident light amount.

As schematically shown in FIG. 3A, the initial state of a low incident light amount shows that the smart watch 1 is not worn on the arm. When the smart watch 1 is worn on the arm, the incident light amount becomes large due to the reflection from the arm. The incident light amount in this case changes like the pulse over time according to blood flow in blood vessels.

That is, in the arm of the user, the amount of green color light absorbed by hemoglobin in the blood periodically increases and decreases according to the amount of blood in the blood vessel increasing and decreasing according to the heartbeat. The pulse sensor 182 measures the increase and the decrease and specifies the cycle. With this, the pulse can be measured. The pulse sensor 182 calculates an average pulse rate for a certain period and outputs the result.

The incident light amount obtained in this way may actually be different depending on the state of the user's arm. The state of the user's arm may be a state that changes in a short span according to the state of the use of the muscles such as the posture of the arm, that is, whether the arm is raised or lowered, whether the arm is placed on a desk, or whether something is being held. The state may also be a state that does not change in a short span such as a degree of sunburn of the skin.

If the incident light amount does not largely increase when the smart watch 1 is worn depending on the above states, the difference of the incident light amount from when the smart watch 1 is not worn does not become large. If the standard value Lth1 is set as a standard value for determining whether the smart watch 1 is worn or not assuming that there is a large change in the incident light amount as in a measured example M2, there may be cases such as a measured example M1 in which the determination of whether the smart watch 1 is worn cannot be performed accurately depending on the state of the arm. Therefore, preferably, as the standard to determine whether the smart watch 1 is worn or removed, it is preferable to determine a value (standard value Lth1) that is near a minimum as the incident light amount when the smart watch 1 is worn.

FIG. 3A shows a situation assuming that the incident light amount is constant when the smart watch 1 is not worn. However, the position where the smart watch 1 is placed when the smart watch 1 is not worn is different depending on the user, and such incident light amount when the smart watch 1 is not worn may influence the setting of the standard value.

As shown in FIG. 3B, by measuring the incident light amount in each of the plurality of postures in a state wearing the smart watch 1, the posture in which the smallest incident light amount can be achieved is specified. Here, the incident light amount is at the minimum when the user is in posture E.

The posture of the arm in which such minimum incident light amount can be obtained is basically the same among many people, but the positive value may be different among each person depending on the degree of sunburn. The posture in which the minimum incident light amount can be obtained is different for each part that the electronic device is worn in electronic devices that are worn in parts other than the arm. Therefore, in the smart watch 1, by actually measuring the incident light amount before actual use or during use, an appropriate determination standard value (first standard value) regarding determination of whether the smart watch 1 is worn or not is set for each part.

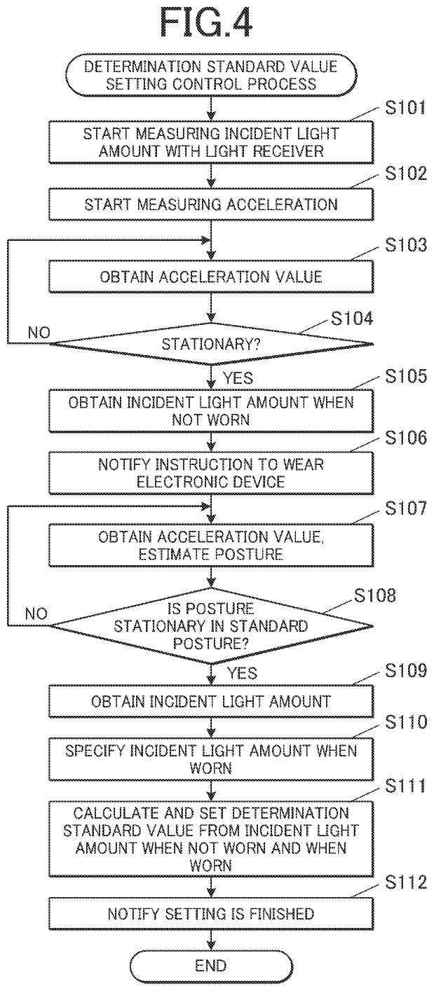

FIG. 4 is a flowchart showing a control procedure by the CPU 11 when executing the determination standard value setting control process in the smart watch 1 of the present embodiment.

Such determination standard value setting control process is a determination standard setting method according to the present embodiment. For example, the determination standard value setting control process starts by reading a program 131 when the CPU 11 detects that an input operation requesting the process to be executed is received by the operation receiver 15.

In such determining standard value setting control process, a standard posture in which it is assumed that the minimum incident light amount can be obtained is determined in advance, and the standard posture is stored in the standard information 132 of the storage 13.

The CPU 11 starts measuring the incident light amount by the light receiver 1822 (step S101). The CPU 11 starts measuring the acceleration by the acceleration sensor 181 (step S102).

The CPU 11 obtains acceleration values at appropriate intervals (step S103). The CPU 11 determines whether the smart watch 1 is in a stationary state based on the acceleration value (measured result) (step S104). The stationary state here is not a momentary pause, but is a state in which the acceleration other than the gravitational acceleration is sufficiently small continuously for a certain amount of time. If it is determined that the smart watch 1 is not in the stationary state (“NO” in step S104), the process by the CPU 11 returns to step S103.

If it is determined that the smart watch 1 is the stationary state (“YES” in step S104), the CPU 11 obtains the incident light amount to the light receiver 1822 as the incident light amount when the smart watch 1 is not worn (step S105).

The CPU 11 controls the notification operator 16 and the display 14 to perform a notification operation to the user wearing the smart watch 1 (step S106). Here, the CPU 11 may control the notification operation 16 and the display 14 to perform the notification operation to the user so that the user performs the input operation on the operation receiver 15 to select whether the smart watch 1 is worn on the left arm or the right arm.

The CPU 11 obtains the acceleration value (based on the measured result of the acceleration sensor 181) and estimates (determines) a posture of the arm based on a gravitational acceleration direction and the like (step S107, determiner). The CPU 11 determines whether the arm is stationary (maintained) in the predetermined standard posture (step S108). For example, the determination of the standard posture may be performed by whether the angle of the arm is within a preset allowable range of a shift. If the posture cannot be uniquely determined by only the acceleration in the stationary state, the CPU 11 may follow the change in the acceleration related to the movement of the arm. Alternatively, the CPU 11 may simply determine that the user correctly moved the arm according to the notification operation. If it is determined that the arm is not stationary in the standard posture (“NO” in step S108), the process by the CPU 11 returns to step S107.

If it is determined that the arm is stationary in the standard posture (“YES” in step S108), the CPU 11 obtains the incident light amount to the light receiver 1822 (step S109). The CPU 11 specifies the incident light amount when the smart watch 1 is worn based on the tendency of the change of the incident light amount for a certain amount of time (step S110). As described above, the incident light amount when the smart watch 1 is worn changes periodically according to a change in the blood flow. Therefore, for example, the incident light amount when the smart watch 1 is worn may be an average value of a plurality of cycles or may be a minimum value. Alternatively, after removing the noise from the incident light amount of a certain amount of time by the low pass filter, a minimum value or a minimum line of the incident light amount may be obtained.

The processes in steps S108 to S110 are the setting means according to the present embodiment.

The CPU 11 calculates a determination standard value to determine a worn or not worn state based on the incident light amount when the smart watch 1 is not worn and the incident light amount when the smart watch 1 is worn. The CPU 11 stores and sets the determination standard value in the standard information 132 of the storage 13 (step S111). For example, the determination standard value may be an average value of the incident light amount when the smart watch 1 is worn and not worn or may be a weighted average slightly weighted on either side.

The CPU 11 controls the notification operator 16 and the display 14 to perform the notification operation showing that the setting of the determination standard value is complete (step S112). Then, the CPU 11 ends the determination standard value setting control process.

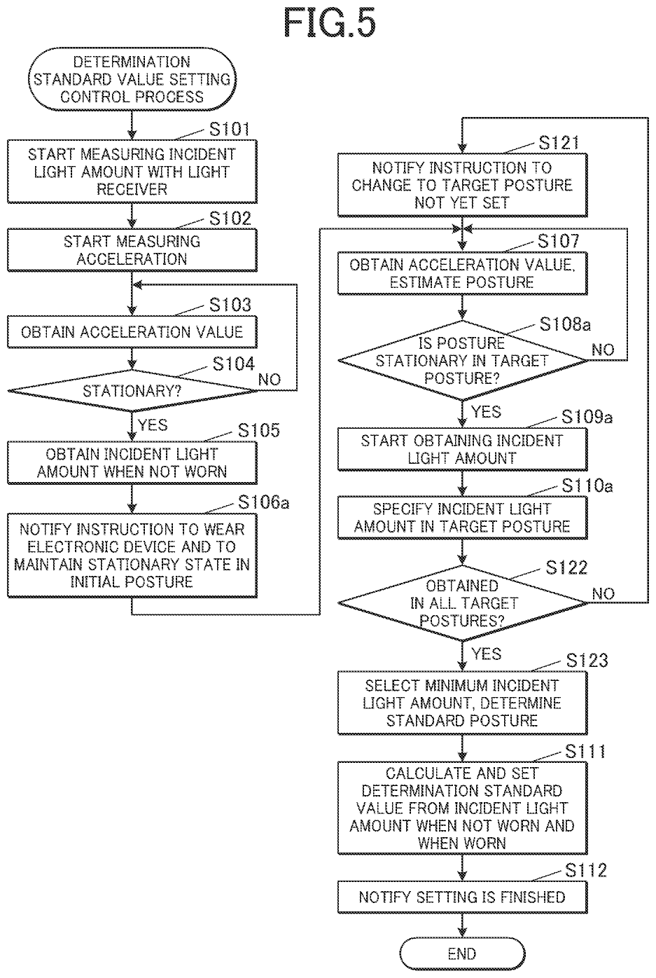

FIG. 5 is a flowchart showing another example of the determination standard value setting control process.

In this determination standard value setting control process, the processes of step S106 and steps S108 to S110 included in the process as shown in FIG. 4 are replaced with step S106a and steps S108a to S110a. Moreover, the processes in steps S121 to S123 are added. The other processes are the same among the examples. The same reference numerals are applied to the same processing contents and the detailed description is omitted.

This determination standard value setting control process is different from the process shown in FIG. 4 in that the standard posture is not determined in advance.

After step S105, the CPU 11 controls the notification operator 16 and the display 14 to perform the notification operation to maintain a stationary state in the initial posture while wearing the smart watch 1 (step S106a). Then, the process by the CPU 11 proceeds to step S107.

After the process in step S107, the CPU 11 determines whether the user is placing the arm stationary in a specified target posture based on the acceleration measured value (step S108a). If it is determined that the arm is not stationary in the target posture (“NO” in step S108a), the process by the CPU 11 returns to step S107.

If it is determined that the user's arm is stationary in the target posture (“YES” in step S108a), the CPU 11 starts obtaining the incident light amount (step S109a). The CPU 11 specifies the incident light amount in the target posture based on the obtained incident light amount (step S110a). Similar to the example shown in FIG. 4, the CPU 11 considers the change in the incident light amount while the smart watch 1 is worn and specifies and calculates the average value and the minimum value of the change and the minimum line based on the minimum value of each cycle.

The CPU 11 determines whether the incident light amount is specified and obtained for all of the plurality of target postures (step S122). The plurality of target postures (plurality of postures) may be stored in the storage 13 in advance. If it is determined that the incident light amount is not obtained in all of the target postures (there are target postures in which the amount is not obtained) (“NO” in step S122), the CPU 11 controls the notification operator 16 and the display 14 to perform the notification operation to instruct the user to change the posture to the target posture in which the amount is not set (step S121). Then, the process by the CPU 11 proceeds to step S107.

In the determining process in step S122, if it is determined that the incident light amount is specified and obtained in all of the target postures (“YES” in step S122), the CPU 11 compares the incident light amount in each target posture and selects the smallest incident light amount. The CPU 11 determines (specifies) the target posture with which the minimum incident light amount is obtained as the standard posture (step S123). Then, the process by the CPU 11 proceeds to step S111.

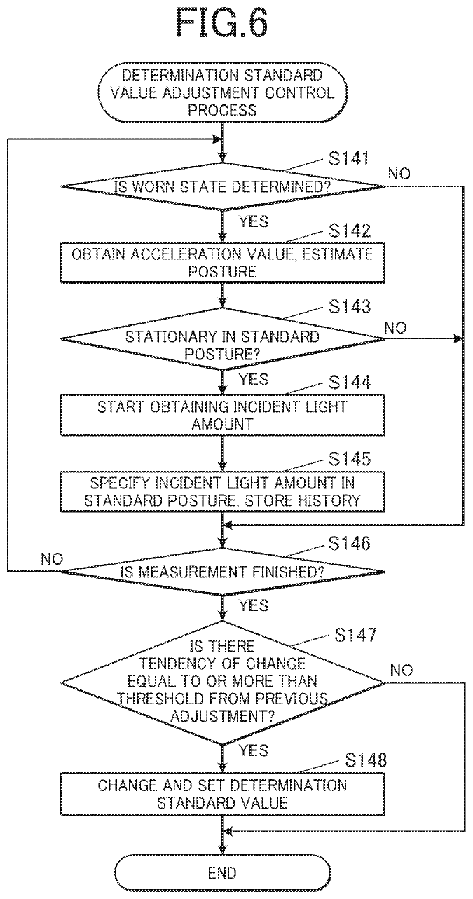

FIG. 6 is a flowchart showing a control procedure executed by the CPU 11 in the determination standard value adjustment control process performed by the smart watch 1. This determination standard value adjustment control process starts together with the start of the determination of whether the smart watch 1 is worn and the measuring of the pulse. The start of the process may be limited to when a measurement of a specific activity starts. In this case, in response to the start of measuring the pulse, the measuring of the acceleration sensor 181 and the pulse sensor 182 (worn/removed sensor) starts in another process.

The CPU 11 performs the determination of whether the smart watch 1 (own device) is worn on the arm of the user based on the standard value and the measured value of the incident light amount (step S141). Alternatively, the comparison between the standard value and the measured value of the incident light amount may be performed in an external dedicated circuit, and the CPU 11 may simply obtain the results. If it is determined that the smart watch 1 is not worn on the arm (“NO” in step S141), the process by the CPU 11 proceeds to step S146.

If it is determined that the smart watch 1 is worn on the arm (“YES” in step S141), the CPU 11 obtains the acceleration value from the acceleration sensor 181 and estimates the posture of the arm (step S142). The CPU 11 determines whether the arm is in a stationary state in the standard posture (step S143). If it is determined that the arm is not in the stationary state in the standard posture (“NO” in step S143), the process by the CPU 11 proceeds to step S146.

If it is determined that the arm is stationary in the standard posture (“YES” in step S143), the CPU 11 starts obtaining the incident light amount incident to the light receiver 1822 (step S144). The CPU 11 specifies the incident light amount in the standard posture, and stores the amount as history information in the storage 13 (step S145). Then, the process by the CPU 11 proceeds to step S146.

When the process advances to step S146, the CPU 11 determines whether there is an operation to end the measurement of the pulse (step S146). If it is determined that there is no operation to end the measurement (“NO” in step S146), the process by the CPU 11 returns to step S141. Basically, when the measurement of the pulse always continues, instead of determining whether there is an operation to end the measurement, for example, a determination regarding a periodic time elapse such as whether the time passed a specific time may be performed.

When it is determined that there is an operation to end the measurement (“YES” in step S146), the CPU 11 determines whether there is a tendency of change (change over time) with the change equal to or more than the threshold after adjusting the determination standard value (step S147). For example, such change that cannot be overlooked is assumed to be a middle to long term such as change in the degree of sunburn and change in how much muscles are attached, but the change is not limited to the above.

If it is determined that there is no tendency of change with the change that is equal to or more than the threshold value from the previous adjustment (“NO” in step S147), the CPU 11 ends the determination standard value adjustment control process. If it is determined that there is tendency of change with the change that is equal to or more than the threshold from the previous adjustment, the CPU 11 changes (adjusts) and sets the determination standard value based on the tendency of change (step S148). Then, the CPU 11 ends the determination standard value adjustment control process.

Opposite of the above, when the incident light with a larger amount of light than the maximum incident light assumed according to the emitted light is measured, it is possible to determine that the smart watch 1 is not worn on the arm. For example, the incidence of such light amount may occur by direct sunlight or light reflected on a surface with a high reflection rate such as a white desk or wall. In the part where the smart watch 1 is worn such as the arm, there is diffuse reflection and light absorption. In the smart watch 1, an incident light amount that is sufficiently higher than the maximum assumed incident light amount considering the above may be set as another determining standard value (second standard value) and may be used in the determination of whether the smart watch 1 is worn or not. The incident light amount that should be actually considered may be obtained by measuring in advance the value when the smart watch 1 is placed on a white desk facing a preset direction in a laboratory or the like. The smart watch 1 may be placed so that the base simply faces the desk surface or may be placed so that a certain direction from a side surface of the case 3 such as a 3 o'clock direction comes into contact with the desk surface. The second standard value is set to be smaller than the obtained actual measured value and sufficiently larger than the maximum assumed incident light amount, and the value is stored in the storage 13 before shipping the product. More roughly, the second standard value may be equal to the emitted light amount of the light emitter 1821 or a coefficient multiple of the emitted light amount, the coefficient multiple being larger than 1. Alternatively, the maximum incident light amount may be experimentally obtained and used instead of the maximum assumed incident light amount. The maximum incident light amount may be selected with a second standard posture in the process of step S123 shown in FIG. 5. The second standard value may be adjusted to a value obtained by multiplying this maximum incident light amount by a coefficient larger than 1, or may be a weighted average at a certain ratio between the maximum incident light amount and the actual measured value in the laboratory.

In addition to the above, even if the value is within the normal range of the incident light amount, there may be exceptions such as the assumed change according to the pulse cannot be achieved (the reflection rate of the surface reflecting the light such as the desk is not high or the smart watch 1 is placed facing a direction in which it is difficult for the reflected light to enter). Regarding such exceptions, a determination that the smart watch 1 is not worn may be made based on the other suitably determined determination standards.

As described above, the smart watch 1 which is a wearable electronic device according to the present embodiment includes the light emitter 1821 that emits light with a certain wavelength from the surface (bottom surface) in contact with the body of the user when the device is worn, the light receiver 1822 that measures the incident light amount of the light with the certain wavelength, the acceleration sensor 181 that measures the posture of the part where the smart watch 1 is worn, and the CPU 11. The CPU 11 determines (estimates) the posture of the part where the smart watch 1 is worn based on the result measured by the acceleration sensor 181. The CPU 11 sets the determination standard value (first standard value) to determine whether the smart watch 1 is worn or not based on the incident light amount when it is determined that the part where the smart watch 1 is worn is in the standard posture. As described above, the determination standard value to detect whether the smart watch 1 is worn or taken off is determined with the standard posture suitable for each user. Therefore, it is possible to reduce the possibility of mistaking the determination of whether the smart watch 1 is worn or not, that is, the possibility of determining that the smart watch 1 is not being worn even when the smart watch 1 is actually worn.

That is, the reflected amount of light in a state that the smart watch 1 is worn is different depending on conditions such as the posture when the smart watch 1 is worn. If the determination standard is determined uniformly, it is not always possible to accurately determine whether the smart watch 1 is worn or not. However, according to the present disclosure, it is possible to cope with such cases and the determination of whether the smart watch 1 is being worn can be performed accurately.

Moreover, the standard posture can be determined in advance. The posture suitable for determining the determination standard value and the posture in which the incident light amount tends to become small is basically determined regardless of the individual. Therefore, by determining the determination standard value based on the incident light amount in this posture, it is possible to enhance the accuracy of determining whether the smart watch 1 is worn using the light emitter 1821 and the light receiver 1822 in the smart watch 1.

The smart watch 1 includes the operation receiver 15. When the CPU 11 receives the request to set the determination standard value with the operation receiver 15, the CPU 11 performs the process regarding the setting of the determination standard value. That is, when the user actively starts the processing at the timing desired by the user, the smart watch 1 is able to easily measure and obtain the incident light amount at the appropriate standard posture.

After the determination standard value is set, the CPU 11 determines whether the part where the smart watch 1 is worn is in the standard posture in the state that the determination of whether the smart watch 1 is worn is performed. The CPU 11 obtains the incident light amount if it is determined that the part where the smart watch 1 is worn is in the standard posture in a state that the smart watch 1 is worn in the worn part. The CPU 11 adjusts the determination standard value based on the change over time of the obtained incident light amount.

During the actual operation of determining whether the smart watch 1 is being worn, the user may be in various postures. Therefore, the CPU 11 is able to detect a convenient posture and obtain the incident light amount when the user is in that posture. Such incident light amount may change depending on the degree of sunburn even when the user is in the same posture. Therefore, by adjusting the determination standard value while observing the middle to long term trend, it is possible to continue accurate determination of whether the smart watch 1 is being worn.

The CPU 11 may obtain the incident light amount of the part where the smart watch 1 is worn in a plurality of postures, and specify the standard posture by comparing the obtained incident light amount. With this, it is possible to determine the optimum standard posture for each user. Therefore, the CPU 11 is able to determine the determination standard value for more accurately determining whether the smart watch 1 is worn suitably reflecting the differences among individuals.

The standard posture may be the posture in which the incident light amount becomes minimum among the plurality of postures. When determined for each individual, by simply determining the direction that the incident light amount becomes the minimum as the standard posture, it is possible to efficiently reduce the possibility of erroneously determining whether the smart watch 1 is worn for each individual.

The smart watch 1 includes the notification operator 16 that performs the notification operation to the user. The plurality of postures are determined in advance. The CPU 11 controls the notification operator 16 to perform the notification operation to specify to the user to take each of the plurality of postures, and obtains the incident light amount of the plurality of postures. That is, when each user sets the determination standard value, by appropriately guiding the change of the posture by using the notification operator 16, the smart watch 1 is able to smoothly set the determination standard value.

When it is determined that each of the plurality of postures is maintained based on the measurement by the acceleration sensor 181, the CPU 11 may obtain the incident light amount for each posture. Since a difference can be seen depending on how the muscles are used in an active state, by obtaining the incident light amount in a state that each posture is maintained and kept stationary, the smart watch 1 is able to more suitably determine the determination standard value.

According to the smart watch 1, the second standard value larger than the first standard value may be compared with the incident light amount. The CPU 11 determines that the smart watch 1 is not worn (taken off) when the incident light amount is larger than the second standard value. Such second standard value may be stored in a usable state in the storage 13 so that the smart watch 1 is able to accurately perform the determination of whether the smart watch 1 is being worn.

The determination standard setting method according to the present embodiment is a method to determine whether the wearable electronic device (smart watch 1) is being worn. The wearable electronic device includes a light emitter 1821 that emits light with a certain wavelength from the surface worn on the body of the user, the light receiver 1822 that measures the incident light amount of the light with the certain wavelength, and the acceleration sensor 181 that measures the posture of the part where the smart watch 1 is worn. According to such determination standard setting method, the posture of the part where the device is worn is determined based on the measured result by the acceleration sensor 181, and based on the incident light amount when it is determined that the part where the device is worn is in the standard posture, the determination standard value (first standard value) to determine whether the smart watch 1 is worn or not is set. Since the determination standard value is determined by the actually detected value of the incident light amount when the part where the device is worn is in the standard posture, according to this determination standard setting method, it is possible to reflect the variation among the users and to set the determination standard value for determination of whether the smart watch 1 is worn or not so that erroneous determination hardly occurs.

The program 131 according to the determination standard setting method is installed in the computer and executed. With this, it is possible to perform the determination of whether the smart watch 1 is worn easily and accurately based on the incident light amount to the light receiver 1822.

The present disclosure is not limited to the above embodiments and various changes are possible.

For example, according to the above embodiment, the incident light amount when the smart watch 1 is not worn is measured but this process may be omitted.

According to the present embodiment, the light emitter 1821 and the light receiver 1822 of the pulse sensor 182 are used to also function as the worn/removed sensor and are used to set the standard value of such sensor, but the present embodiment is not limited to the above. The configuration other than the light emitting of the pulse sensor may be used. The pulse sensor does not have to be provided in the device. The light emitter and the light receiver of another component may be used as the worn/removed sensor, or a completely independent worn/removed sensor may include a light emitter and a light receiver.

When the part where the wearable electronic device (smart watch 1) is worn is not limited to one part, the determination standard value may be set for each part where the device is worn.

The setting of the determination standard value does not have to start by the user operation with the intention of setting the value. For example, when the pulse measurement is started in a state in which the determination standard value is not set, the process may automatically proceed to the setting operation of the determination standard value.

The adjustment of the determination standard value according to the change over time may be performed without using the measured value in normal use as described above. That is, a request to the user may be made at a predetermined interval to reset the determination standard value with the same procedure as the initial setting.

The influence of the change in the cycle due to the blood flow that can be seen in the incident light amount when the smart watch 1 is worn may be removed or considered with the method other than the above.

When a plurality of postures are determined as the standard postures in advance, the standard posture does not have to be the posture in which the incident light amount becomes the minimum in many cases. For example, the standard posture may be determined with the standard that the possibility of the incident light amount becoming large is small.

According to the present embodiment, the measured plurality of postures are stored in advance but the embodiment is not limited to the above. The notification operator 16 and the display 14 may perform the notification operation to gradually change the posture from the initial position in a direction to reduce the incident light amount.

The posture measurement does not have to be measured by the acceleration sensor 181 and may be measured with another component in addition to the acceleration sensor 181. For example, a geomagnetic sensor, a gyro sensor, an inclination sensor or the like may be used or used in combination.

The wearable electronic device does not have to be worn on the arm. The device may be worn on an upper arm, a torso, or a leg. Such wearable electronic device does not have to be a device for the purpose of measuring the vitals, activities and the like of the user.

The worn/removed sensor does not have to perform the determination of whether the smart watch 1 is worn or not by only the set including the light emitter and the light receiver. The determination may be performed together with the determination of whether the smart watch 1 is worn or not by using the change in the acceleration by the acceleration sensor 181 or the change in the electric resistance (capacitance) in the contact surface such as the back cover 2. In this case, the determination standard value may be determined on the premise that the above will also be used.

According to the above embodiment, the notification operator 16 and the display 14 guide the posture, but other methods can be employed. For example, the specific posture may be shown in an instruction manual or an online instruction manual can be referred to from other devices such as a smart phone. The smart watch 1 may be configured to be able to output audio and a guidance may be performed by the audio.

According to the determination standard value adjustment control process shown in FIG. 6, the determination of the tendency of the change is collectively performed when the actual determination operation ends. However, in the determination standard value adjustment control process, the determination of the tendency of the change may be performed when the history is obtained to some degree even if the determination operation is in progress, and the determination standard value may be changed and set.

According to the above description, the storage 13 is provided as an example of a nonvolatile memory such as a flash memory or the like which is a computer-readable storage medium that stores the program 131 related to the control of the determination standard value setting according to the present disclosure. However, the storage is not limited to the above. As the computer-readable storage medium, a nonvolatile memory such as a MRAM, etc., and a portable storage medium, such as a CD-ROM, a DVD disk, etc. may also be used. A carrier wave is also applied to the present disclosure as the medium to provide data of the program according to the present disclosure through the communication lines.

Other specific configurations, contents and procedures of the operation, and the like as described in the above embodiments may be suitably changed without leaving the scope of the present disclosure.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-152140, filed Sep. 26, 2022 which is hereby incorporated by reference wherein in its entirety.

What is claimed is:

1. A wearable electronic device comprising:
a light emitter that emits light with a certain wavelength toward a body while the device is worn on the body;
a light receiver that measures an incident light amount of the light with the certain wavelength;
a posture measurer that measures a posture of a part of the body where the device is worn; and
one or more processors,
wherein the processor is configured to,
determine a posture of the part of the body where the device is worn based on a measured result measured by the posture measurer, and
set a first standard value for determining whether the device is worn on the body or not based on the incident light amount in a case that the part of the body where the device is worn is in a standard posture.

2. The wearable electronic device according to claim 1, wherein the standard posture is predetermined.

3. The wearable electronic device according to claim 1, further comprising an operation receiver,
wherein the processor is configured to perform a process regarding a setting of the first standard value in a case that the operation receiver receives a request to set the first standard value.

4. The wearable electronic device according to claim 1,
wherein the processor is configured to,
after setting the first standard value,
determine whether the device is worn on the body or not, determine whether the part where the device is worn is in the standard posture and obtain the incident light amount in a case that it is determined that the part where the device is worn is in the standard posture, and
adjust the first standard value based on a change over time in the obtained incident light amount.

5. The wearable electronic device according to claim 1, wherein the processor is configured to obtain the incident light amount of each of a plurality of postures of the part where the device is worn, compare the obtained incident light amount of the plurality of postures, and specify the standard posture.

6. The wearable electronic device according to claim 5, wherein the standard posture is a posture in which the incident light amount becomes minimum among the plurality of postures.

7. The wearable electronic device according to claim 5, further comprising a notification operator that performs a notification operation to a user,
wherein,
the plurality of postures are predetermined, and
the processor is configured to control the notification operator to perform a notification operation to indicate to the user each posture among the plurality of postures and is configured to obtain the incident light amount while the user is in each of the plurality of postures.

8. The wearable electronic device according to claim 5, wherein the processor obtains the incident light amount for each of the plurality of postures in a case that it is determined that a user is maintaining each of the plurality of postures based on the measurement by the posture measurer.

9. The wearable electronic device according to claim 1, wherein the processor compares the incident light amount with a second standard value larger than the first standard value, and in a case that it is determined that the incident light amount is larger than the second standard value, the processor determines that the device is not worn.

10. A determination standard setting method that determines whether a wearable electronic device is worn or not, the wearable electronic device including a light emitter that emits light with a certain wavelength toward a body while the device is worn on the body; a light receiver that measures an incident light amount of the light with the certain wavelength; and a posture measurer that measures a posture of a part of the body where the device is worn, the determination standard setting method comprising:
determining a posture of the part of the body where the device is worn based on a measured result measured by the posture measurer, and
setting a first standard value for determining whether the device is worn on the body or not based on the incident light amount in a case that the part of the body where the device is worn is in a standard posture.

11. The determination standard setting method according to claim 10, further comprising,
after setting the first standard value, determining whether the device is worn on the body or not, determining whether the part where the device is worn is in the standard posture, and obtaining the incident light amount in a case that it is determined that the part where the device is worn is in the standard posture, and
adjusting the first standard value based on a change over time in the obtained incident light amount.

12. The determination standard setting method according to claim 10, further comprising obtaining the incident light amount of each of a plurality of postures of the part where the device is worn, comparing the obtained incident light amount of the plurality of postures, and specifying the standard posture.

13. The determination standard setting method according to claim 12, wherein the standard posture is a posture in which the incident light amount becomes minimum among the plurality of postures.

14. The determination standard setting method according to claim 12, wherein,
the wearable electronic device further includes a notification operator that performs a notification operation to a user,
the plurality of postures are predetermined, and
the notification operator performs a notification operation to indicate to the user each posture among the plurality of postures to be taken by the user and the incident light amount while the user is in each of the plurality of postures is obtained.

15. A non-transitory computer readable storage medium, storing a program executable by one or more processors in a wearable electronic device, the wearable electronic device including a light emitter that emits light with a certain wavelength toward a body while the device is worn on the body;
a light receiver that measures an incident light amount of the light with the certain wavelength; and a posture measurer that measures a posture of a part of the body where the device is worn, the program causing the one or more processors to perform:
determining a posture of the part of the body where the device is worn based on a measured result measured by the posture measurer, and
setting a first standard value for determining whether the device is worn on the body or not based on the incident light amount in a case that the part of the body where the device is worn is in a standard posture.

16. The storage medium according to claim 15, wherein the program further causes the one or more processors to perform,
after setting the first standard value, determining whether the device is worn on the body or not, determining whether the part where the device is worn is in the standard posture, and obtaining the incident light amount in a case that it is determined that the part where the device is worn is in the standard posture, and
adjusting the first standard value based on a change over time in the obtained incident light amount.

17. The storage medium according to claim 15, wherein the program further causes the one or more processors to perform obtaining the incident light amount of each of a plurality of postures of the part where the device is worn, comparing the obtained incident light amount of the plurality of postures, and specifying the standard posture.

18. The storage medium according to claim 17, wherein the standard posture is a posture in which the incident light amount becomes minimum among the plurality of postures.

19. The storage medium according to claim 17, wherein,
the wearable electronic device further includes a notification operator that performs a notification operation to a user,
the plurality of postures are predetermined, and
the program further causes the one or more processors to perform controlling of the notification operator to perform a notification operation to indicate to the user each posture among the plurality of postures and obtaining the incident light amount while the user is in each of the plurality of postures.
